(19) United States
(12) Patent Application Publication
(10) Pub. No.: US 2017/0172660 A1
(43) Pub. Date: Jun. 22, 2017

(54) IRRADIATION UNIT FOR PROVIDING RADIATION PULSES FOR IRRADIATING A SKIN SURFACE AND METHOD FOR OPERATING AN IRRADIATION UNIT

(71) Applicant: OSRAM GmbH, Munich (DE)

(72) Inventors: Oliver Mehl, Berlin (DE); Holger Laabs, Berlin (DE)

(21) Appl. No.: 15/384,375

(22) Filed: Dec. 20, 2016

(30) Foreign Application Priority Data
Dec. 21, 2015 (DE) 10 2015 226 377.0

(51) Int. Cl. A61B 18/20; A61N 5/06

(52) U.S. Cl. CPC A61B 18/203; A61N 5/0616; A61N 2005/0626; A61N 2005/0651; A61N 2005/067

(57) ABSTRACT

An irradiation unit for providing radiation pulses for irradiating a skin surface is provided. The irradiation unit includes a light source unit configured to provide the radiation pulses with a specifiable pulse duration, with a specifiable pulse height and with a specifiable temporal pulse spacing. The light source unit is furthermore configured to illuminate a region of the skin surface of a predetermined size at a predetermined distance from the light source unit in a main radiation direction of the light source unit. The light source unit includes at least one solid-state light source. The irradiation unit further includes a sensor unit and a control device for driving the at least one solid-state light source.

### CROSS-REFERENCE TO RELATED APPLICATION

This application claims priority to German Patent Application Serial No. 10 2015 226 377.0, which was filed Dec. 21, 2015, and is incorporated herein by reference in its entirety.

### TECHNICAL FIELD

Various embodiments relate generally to an irradiation unit for providing radiation pulses for irradiating a skin surface, e.g. for epilation, and from a method for operating an irradiation unit, wherein the irradiation unit has a light source unit which is configured to provide the radiation pulses with a specifiable pulse duration, with a specifiable pulse height and with a specifiable temporal pulse spacing. The light source unit is furthermore configured to illuminate a region of the skin surface of a predetermined size at a predetermined distance from the light source unit in a main radiation direction of the light source unit.

### BACKGROUND

Irradiation units having IPL (intense pulsed light) light sources, for example for hair removal, are known from the prior art. Xenon gas-discharge lamps are typically used here in pulsed operation with an emission spectrum of near ultraviolet to near infrared, but also laser light sources and light emitting diode (LED) light sources are used. LED light sources can emit radiation in the range of about 550 nm to about 1200 nm. The absorption of the light pulses in the skin is here substantially determined by water, hemoglobin and melanin. During hair removal, the emphasis is on melanin absorption, wherein melanin occurs both in the skin and in hair follicles. In order to achieve optimum action, an energy introduction into the hair root that is as high as possible is necessary without damaging the skin. The preferred emission wavelength here depends on the skin type, wherein longer wavelengths are typically more suitable for dark skin types. Since xenon gas-discharge lamps have a very wide emission spectrum, longpass filters are usually used to suppress the emission of wavelengths below approximately 600 nm. Moreover, typically the area to be treated is divided into segments for hair removal, which correspond to the size of the treatment head, which is referred to as the applicator. By sequentially moving the applicator, a specific areal energy density is introduced into each segment. This procedure is illustrated in FIG. 1.

FIG. 1 shows a schematic of an area 10 to be treated, which is divided into individual segments 10a of identical size, of which only one is provided with a reference sign by way of example. The size of a segment 10a here corresponds to the size of the applicator. The latter is placed on a segment 10a of the area to be treated or is held at a defined distance therefrom, whereupon one or more light pulses with specifiable pulse duration, pulse spacing and intensity are generated, which ensure the corresponding energy introduction into the segment 10a. Subsequently, the applicator is moved to a next segment 10a, and again one or more light pulses are generated. This manual and successive movement of the applicator is also referred to as stitching.

A disadvantage of the known irradiation units for hair removal is that owing to the use of filters, the energy losses are very high, with the result that the energy efficiency of such an irradiation unit is significantly reduced. Furthermore, the discharge lamps used are only somewhat variable in terms of their temporal discharge behavior, or only with great outlay. A local variation of the energy dose within the treatment field is moreover not possible either. In addition, the methods for hair removal are very complicated, because the area to be treated must be divided into small regions which are illuminated individually and successively by manually moving the applicator. This manual stitching is very laborious and uncomfortable for a user. In addition, the size of these regions is determined by the size of the discharge lamp and the reflector used, with the result that once again there is little flexibility in terms of adapting to the areas to be treated. Laser light sources are expensive and are subject to strict safety regulations, which makes handling complex.

### SUMMARY

An irradiation unit for providing radiation pulses for irradiating a skin surface is provided. The irradiation unit includes a light source unit configured to provide the radiation pulses with a specifiable pulse duration, with a specifiable pulse height and with a specifiable temporal pulse spacing. The light source unit is furthermore configured to illuminate a region of the skin surface of a predetermined size at a predetermined distance from the light source unit in a main radiation direction of the light source unit. The light source unit includes at least one solid-state light source. The irradiation unit further includes a sensor unit and a control device for driving the at least one solid-state light source.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 shows a schematic of an area to be treated with individual segments for illustrating a method for conventional hair removal;

FIG. 2 shows a schematic of an irradiation unit for providing radiation pulses having a plurality of individually drivable LEDs according to an embodiment;

FIG. 3a shows a schematic of the irradiation unit having a plurality of LEDs which are arranged in a line according to an embodiment;

FIG. 3b shows a schematic of the irradiation unit having a plurality of LEDs which are arranged in three lines according to an embodiment;

FIG. 3c shows a schematic of the irradiation unit having a plurality of LEDs which are arranged in lines and have different emission wavelengths according to an embodiment;

FIG. 4 shows a schematic of the driving of the individual LEDs of the irradiation unit in the temporal profile according to an embodiment; and

FIG. 5 shows a table for illustrating the connection between pulse duration and areal energy density according to an embodiment.

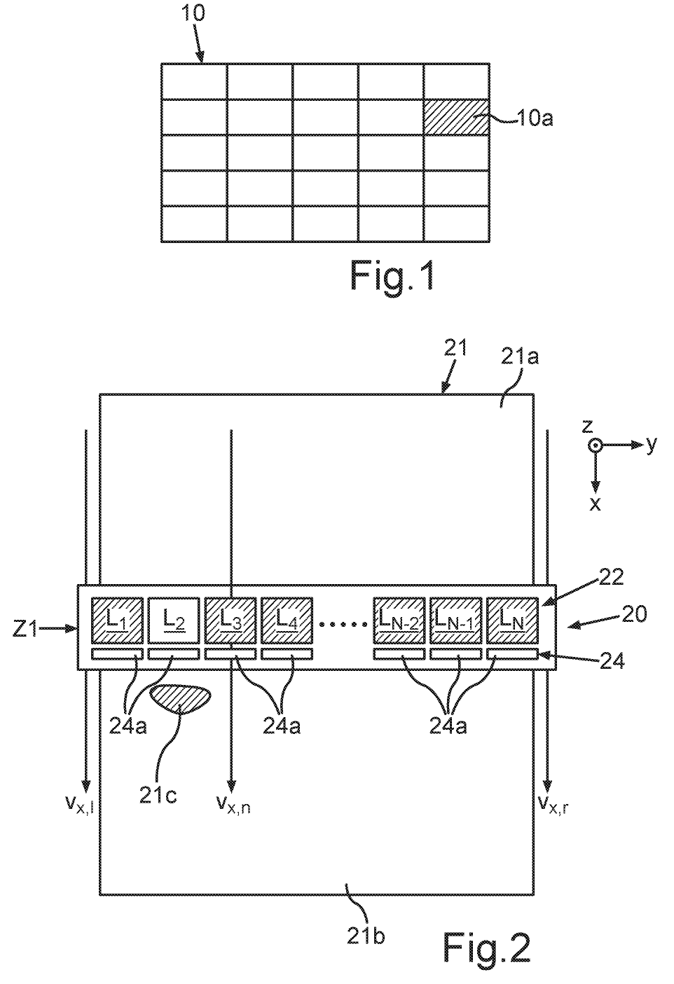

### DESCRIPTION

The following detailed description refers to the accompanying drawings that show, by way of illustration, specific details and embodiments in which the invention may be practiced.

Various embodiments provide an irradiation unit for providing radiation pulses and a method for operating such an irradiation unit, which permit the avoidance of at least one of the above-described disadvantages.

In various embodiments, the light source unit has at least one solid-state light source and the irradiation unit has a sensor unit and a control device for driving the at least one solid-state light source.

Solid-state light sources, such as for example light emitting diodes (LEDs), have a significantly longer lifetime, e.g. as compared to gas-discharge lamps. Additionally, the use of one or more solid-state light sources can significantly increase the energy efficiency. This is because solid-state light sources can be more energy saving than gas-discharge lamps, although in this case, energy efficiency primarily benefits from another property of the solid-state light sources, because the latter can be designed such that they emit in a desired emission range in a very narrow band, e.g. if the light sources are what are known as quantum dot semiconductor light sources. In various embodiments, the entire emission spectrum of the respective solid-state light sources can consequently be used, and no light losses due to typically necessary filters occur. Since the light source unit has at least one, e.g. multiple solid-state light sources, significantly more flexibility with respect to the driving possibilities may be provided, which may permit manifold adaptation possibilities to the respective situation and the intended use. This effect of flexibility may be important e.g. in combination with the sensor unit, because it is thus possible to drive the one or more solid-state light sources in dependence on sensor signals provided by the sensor unit. It is possible by way of the sensor unit to determine situation parameters, such as for example skin properties, skin characteristics, movement speed of the irradiation unit or the like, with the result that the controlling of the one or more solid-state light sources in dependence on these parameters permits particularly good and especially automatic situation adaptation, which significantly increases ease of handling and the functional range of the irradiation unit.

In place of LEDs, it is also possible to use laser diodes as the at least one solid-state light source, e.g. blue laser diodes having a downstream phosphor element, which converts blue primary light at least partially into converted light of a longer wavelength (down conversion) (LARP (laser activated remote phosphor) technology). In dependence on the phosphor used, the converted light can be spectrally narrowband or broadband, for example in the green, yellow, red or infrared spectral range. It is thus possible to generate spectrally narrowband or broadband emission spectra in a targeted manner and to thus achieve improved epilation action. In addition, the at least one solid-state light source can also represent a large-area LED, for example having a large-area chip, e.g. having an extension in at least one direction of one or more centimeters. Moreover, the at least one solid-state light source can also have a plurality of segments, e.g. segments which may be driven individually or in groups, and can be configured for example as an LED having a large-area chip with divided areas which are separately drivable. The embodiments described below for a plurality of the at least one solid-state light source may here be realized in the same way for a solid-state light source having a plurality of segments, as described above.

In one embodiment, the at least one solid-state light source represents an LED, e.g. a high-power LED. It is possible with such LEDs to provide areal energy densities in the range of 0.1 to 100 J/cm², e.g. between 0.8 J per square centimeter and 64 J per square centimeter, with pulse durations of between 1 and 1000 milliseconds, e.g. between 5 and 400 milliseconds. The light source unit can also have a plurality of the at least one solid-state light source, wherein the plurality of solid-state light sources is also arranged along at least one row and/or column. By way of example, an LED array having a plurality of rows and columns can thus be provided. The embodiment of these rows and/or columns in terms of their length and arrangement with respect to one another is variable extremely flexibly by way of the use of LEDs and permits various embodiments of the irradiation unit, which can be configured to a wide variety of situations, for example for the specific treatment of very small areas. In various embodiments, solid-state light sources can have significantly smaller dimensions than gas-discharge lamps, for example chip dimensions of LEDs having an area of 0.5 to 4 mm² having a square, rectangular or round base area can be provided. For example, rows and/or columns can also be configured such that they are extremely variable in terms of their length and width. In addition, solid-state light sources can also be driven highly variably, which entails particularly great flexibility in the dimensioning of the specifiable pulse durations, radiant power and the pulse spacings.

In one embodiment, the light source unit has a plurality of the at least one solid-state light source, and the control device is configured to drive the solid-state light sources in a specifiable sequence such that the solid-state light sources, e.g. at least two of the solid-state light sources, emit light pulses which are temporally offset from one another. Owing to the sequential operation of the solid-state light sources, for example along a row or a column, the necessary pulse power can be lowered, and the requirements in terms of the power electronics of the irradiation unit with respect to the maximum necessary pulse power or with respect to the maximum current to be provided can thus be significantly reduced. Alternatively, the solid-state light sources can also provide the light pulses simultaneously, which permits faster treatment of a predetermined area. A combination of both control possibilities may also be provided, for example it also is possible for the plurality of solid-state light sources to be combined into light source groups. In that case, the solid-state light sources are driven such that all solid-state light sources of the same group emit a light pulse at the same time, while the emission of the light pulses with respect to the individual groups is offset temporally.

In one embodiment, the control device is configured to control the specifiable pulse duration and/or the specifiable pulse height and/or the specifiable pulse spacing as at least one control parameter of the at least one solid-state light source. The pulse height is the height of the pulse current with which the at least one solid-state light source is operated during the pulse duration. The pulse height furthermore determines the radiant power emitted by the solid-state light source during the pulse duration. Owing to the controllability of the pulse height, the radiant power is thus also controllable. This is true e.g. also for a plurality of solid-state light sources.

This embodiment may allow for particularly good adaptation to the respective situation and requirements. The current pulse height and pulse duration here determine the energy that is provided by a pulse and is thus adaptable individually, for example to the skin color or hair color. The pulse spacing, on the other hand, can be adapted to the treatment speed, as will be explained in more detail below.

In one further embodiment, the light source unit in turn has a plurality of the at least one solid-state light source and moreover a plurality of light source groups, wherein each of the light source groups includes at least one of the plurality of solid-state light sources, wherein the control device is configured to control the control parameter for a respective light source group separately and independently from one another. In various embodiments, the control device can also be configured to control the control parameter for a respective solid-state light source of the plurality of solid-state light sources separately and independently from one another. By way of example, the solid-state light sources can thus be controlled separately in terms of their pulse duration, pulse height or radiant power or specifiable pulse spacing. If they are arranged in a row or in a different spatial structure, this flexible control possibility allows for the emitted energy quantity to be varied along the row or across the spatial structure. This in turn allows a region-wise adaptation of the areal energy density which is output to the surface. This permits, for example, for the emitted energy dose to be adapted to skin inhomogeneities, such as for example in the case of differing pigmentation, for example in the case of a melanocytic nevus in the skin area to be treated. Other examples for location-selective irradiation or reduction or avoidance of the radiant power or energy dose can be damaged skin parts due to injuries, such as cuts or burns, due to hematomas, due to skin diseases such as psoriasis, due to port-wine stain. As an alternative to a possibility of driving each individual LED, it is likewise conceivable for the LEDs to be controllable in groups, with the result that the control logic becomes simplified.

In one further embodiment, the sensor unit is configured to capture a sensor variable which relates to a movement of the irradiation unit relative to the skin surface or alternatively relative to another spatial reference variable. The control unit is here configured for controlling the control parameter in dependence on the captured sensor variable. It is thus possible to control the areal energy density provided to the surface in dependence on the movement over the surface. By way of example, in the case of fast movements over the surface, a higher output can be set than in the case of a slow movement over the surface. As a result, a homogeneous and uniform energy introduction into each surface region is advantageously provided. The areal energy density itself is determined by the specifiable pulse duration and the specifiable radiant power (pulse height), and the number of pulses with which an area is illuminated. For hair removal it is thus no longer necessary for the irradiation unit to be placed segment-by-segment on the area to be treated, as is the case in stitching, and indeed the irradiation unit can be, for example, continuously pushed, rolled, or glided over the surface, while the control device controls the control parameters such that the same areal energy density is automatically introduced into each individual segment. This increases the ease of use enormously and is primarily highly time-efficient. The respectively applied energy introductions or irradiation values can be stored, together with their spatial correlation, in a data memory integrated in the irradiation unit and can be retrieved and can serve, for example, as input variables for a later irradiation treatment.

In the case of one further embodiment, the captured sensor variable represents a movement speed with which the irradiation unit is moved relative to the surface, and/or a path distance over which the irradiation unit is moved relative to the surface. It is also possible to capture both sensor variables or to easily ascertain one from the other, since it is possible to easily calculate the movement speed from the captured path distance and the time taken, or vice versa. For capturing these sensor variables, the sensor unit can have, for example, simple mechanical sensors, such as for example a small wheel which can be placed on the surface and which rotates as the irradiation unit moves over the surface, or optical sensors can also be used, such as for example image sensors, cameras or laser tracking, as in a computer mouse, or the like. These sensors permit particularly easy capturing of the path distance or movement speed and optimum adaptation of the control parameters to the captured path distance or movement speed. If the aim is, for example, to introduce a defined areal energy density into a respective surface segment, where the size of this segment represents the predetermined size of the region that is illuminable by the light source unit, this region can be illuminated with a specifiable number of light pulses, e.g. only one light pulse with a specifiable radiant power and pulse duration, and another pulse may be emitted only once the irradiation unit has been moved across this surface segment to the next adjoining surface segment.

In one further embodiment, in which the light source unit has a plurality of the at least one solid-state light source, the sensor unit is configured to capture the sensor variable with respect to two different locations of the light source unit as a first sensor variable and a second sensor variable, and to ascertain therefrom an associated individual sensor variable for a respective light source group or for a respective solid-state light source. The control device is furthermore configured to control the control parameter of the respective light source group or the respective solid-state light source in dependence on the associated individual sensor variable. By way of example, the movement speed can be captured at two different locations of the light source unit as the first and second sensor variables, and the movement speed can be ascertained for example by way of extrapolation or interpolation as the individual sensor variable for a respective light source or light source group. As a result, speed differences in curved movements over the surface, where for example the solid-state light sources move more slowly on the inside of the curve than the solid-state light sources on the outside of the curve, can advantageously be taken into consideration. The control parameters can then be controlled such that a homogeneous energy introduction per surface area is still ensured.

Moreover, the sensor variable can also be captured at more than just two locations, for example in the region of each individual solid-state light source of a row, or in the region of a column of solid-state light sources, as long as they are arranged in a plurality of rows, which permits even more exact control.

The control device can furthermore be configured to control the pulse spacing in dependence on the captured movement speed such that the pulse spacing decreases as the movement speed increases. The pulse spacing is here the preferred control parameter which is controlled in dependence on the movement speed or the path distance, since in contrast to the pulse duration and pulse height, it is independent of other criteria. The aim of hair removal is to attain heating of the hair roots which is as punctiform as possible. For example, if the pulse durations are too long, energy that is supplied by the radiation flows into the tissue, which brings about undesired skin heating. If pulse durations are too long, e.g. the local temperature is not sufficiently increased either, with the result that the therapeutic action, e.g. the destruction of the hair roots, is lowered or does not take place. If the pulse durations are too short, it may be possible that the temperature required to destroy the hair roots is not reached. For this reason, pulse duration and pulse height and the energy dose which is defined thereby may be calculated such that it is possible to achieve thereby a particularly high effectivity in hair removal. The pulse spacings, on the other hand, can be adapted easily to the movement speed independently of further criteria, with the result that each partial region of the surface can be uniformly irradiated with an energy dose which is defined by the pulse duration and pulse height.

Provision may nevertheless be made according to one embodiment for the control device to be configured to control the pulse duration and/or the radiant power and/or the pulse height in dependence on the captured movement speed such that the radiant energy output per unit time increases as the movement speed increases, e.g. in a manner such that the same radiant energy is supplied to each irradiated surface region of equal size. The pulse duration and the radiant power can likewise be suitably varied at least within specifiable limits to permit uniform energy introduction into the skin surface and to still attain great efficiency in hair removal. In addition, the length of the pulse spacings also has a lower limit, that is to say that in the case of a movement over the surface being too fast, no further reduction of the pulse spacings is possible, with the result that for example an additional increase of the radiant power also permits higher treatment speeds.

However, the treatment speed still has an upper limit, which is why it represents an embodiment, if for the movement speed at least one predetermined limit value is given and the irradiation unit is configured to output a warning signal to a user if the limit value is exceeded, and/or if the control device is configured to switch off the irradiation unit if the limit value is exceeded. It may also be possible here, for example, for two limit values to be specified for the movement speed, such that the irradiation unit first emits a warning signal if the first limit value is exceeded, and the irradiation unit is automatically switched off only if the second limit value, which is higher than the first one, is exceeded. As a result, incorrect uses can be avoided, which could cause, for example, inefficient hair removal due to a movement speed which is too great. It is also possible to indicate to the user, for example by way of a display of the irradiation unit or an LED which illuminates in color, for example green, if the movement speed is within a predetermined range. A user is therefore always informed about the correct use of the irradiation unit.

In one further embodiment, in which the light source unit has a plurality of the at least one solid-state light source, at least one first solid-state light source of the solid-state light sources has a first emission spectrum, e.g. having a first centroid wavelength, and at least one second solid-state light source of the solid-state light sources has a second emission spectrum which differs from the first, e.g. having a second centroid wavelength, which differs from the first. By providing solid-state light sources with different emission spectra, one further control parameter is available, specifically the emission wavelength, which permits optimum adaptation to the respective application. The preferred emission wavelength is here dependent on the skin type and/or the hair color, with e.g. longer wavelengths being more suitable, for example, for dark skin types and leading to higher efficiency. Previous radiation exposure can also be relevant here. By configuring the invention in this way, it becomes possible for the selection of the emission wavelength, e.g. by selecting the LEDs to be operated, to be adapted to the respective skin type or hair type.

Provision may be made for example for the control device to be configured to set an emission wavelength of the light source unit in dependence on a user input that is captured via an operating element of the irradiation unit. By way of example, the user can select a desired emission wavelength which is preferred for his individual skin type and hair type via the operating element. For example, the user can find the recommended emission wavelength in a table for respective hair and skin types and their combinations, and then input it at the operating element. It is likewise conceivable for the user to input his skin and/or hair color at the operating element, and for the irradiation unit to determine the emission wavelength to be selected in accordance with a table stored in a memory and to drive the corresponding LEDs during application. By providing an operating element, a particularly simple and cost-effective adaptation of the emission wavelength to individual user properties is provided.

Alternatively or additionally, however, fully automated adaptation of the emission wavelength to skin and/or hair color by way of the irradiation unit itself may be provided. In this context, one embodiment represents the situation where the sensor unit is configured to determine a color of the surface, in particular a hair color and/or skin color, e.g. also separately for respective partial regions of the surface to be irradiated or of the region of a specific size which is illuminable by the light source unit, wherein the control device is configured to control the control parameter and/or an emission wavelength of the light source unit in dependence on the captured color. For hair removal, radiation with a wavelength of between approximately 550 nm and 1200 nm can be used. Adaptation of the emission wavelength to the respective skin type or hair color type can now also occur automatically therewith. It is also possible for the other control parameters to be controlled in dependence on the captured skin color, e.g. the skin color which is captured in a region-wise manner. This permits for example adaptation, e.g. region-wise adaptation, of the output in the case of detected skin pigments or a melanocytic nevus. This may avoid the introduction of too high an energy dose in the region of local variations in pigmentation. To this end, the control device can be configured to spatially vary the energy dose in dependence on the captured skin color, and for example to reduce in terms of output or switch off individual solid-state light sources in the corresponding region. Provision may also be made for the emission wavelength to be settable manually by a user, while the radiant power, pulse duration and possibly the pulse spacing are set and/or varied in dependence on the skin color captured by the sensor.

In an embodiment, the sensor unit is therefore configured to capture a skin characteristic, e.g. a skin color, of the skin surface, wherein the control device is configured to control the at least one or the multiple solid-state light sources in dependence on the captured skin characteristic for irradiating the region. The skin characteristic can here represent the above-described skin color and/or hair color of the hairs on the skin surface, and also skin color differences with respect to individual regions of the skin surface. The skin characteristic can moreover also represent skin inhomogeneities, such as for example differing pigmentation, for example a melanocytic nevus, a skin injury, such as a cut or burn, a hematoma, skin inhomogeneities due to skin diseases such as psoriasis, and/or a damaged skin part or skin damage, for example due to port-wine stains. Controlling the solid-state light sources in dependence on such captured skin characteristics allows that such skin regions are automatically irradiated with a lower areal energy density or not at all. For example, if the light source unit includes only one solid-state light source, the latter can be switched off or reduced in terms of its output in dependence on the captured skin characteristic, with the result that the region in which the skin characteristic is located is not irradiated or not as strongly. If the light source unit for example includes a plurality of the at least one solid-state light source, the solid-state light sources can be controlled individually or in groups in dependence on the captured skin characteristic, with the result that for example only the partial region in which the skin characteristic is located of the entire region to be irradiated is not irradiated at all or is irradiated less. This means that the user does not need to pay attention during the application on which skin regions he radiates and which ones he leaves out, which significantly improves the ease of use and the safety.

One effect here if the skin characteristic represents a skin characteristic which is located at least partially in the region of the skin surface, wherein the sensor unit is configured to capture the skin characteristic before the irradiation of the region, in terms of time. What is ensured therewith is that the driving of the one or more solid-state light sources can be adapted in a timely manner to the captured skin characteristic.

It is additionally provided here if the sensor unit is configured to capture a position of the skin characteristic within the region and/or to capture a shape of the skin characteristic. In various embodiments, the solid-state light sources can be assigned in each case to a partial region of the region to be irradiated, e.g. by way of the radiation, which is emitted by a respective light source during the emission of a radiation pulse, striking at least mainly the assigned partial region. Due to the localization of skin characteristics in the region to be irradiated, e.g. by determining the position and/or shape thereof, it is then possible to spatially adapt the driving of the solid-state light sources in optimum fashion to the position of the captured skin characteristic.

For example, one further embodiment, in which the light source unit has a plurality of the at least one solid-state light source, makes provision for the control device to be configured to drive the solid-state light sources such that the region is irradiated with an irradiation characteristic, e.g. areal energy density and/or emission wavelength, which varies spatially over the region in dependence on the captured skin characteristic, e.g. in dependence on the position and/or shape. The irradiation characteristic may vary in the irradiated region with the skin characteristic such that the areal energy density at the location or position of the skin characteristic is reduced. By way of example, the solid-state light sources which correspond to one or more partial regions over which the skin characteristic extends may emit no radiation pulse at all, or emit radiation pulses having a greater pulse spacing, smaller pulse height or reduced pulse duration with respect to other solid-state light sources.

Moreover, the sensor unit for capturing the skin color, or generally the skin characteristic, may include a photosensor or a photodetector, for example a photodiode, a photocell, a phototransistor, or the like. With a photosensor it is possible, for example, to capture the light which is emitted by the light source unit and scattered back at the skin surface, and to draw conclusions as to the skin color in dependence on the intensity captured by the photosensor, since skin surfaces of different skin color also have different absorption and reflection properties. Light is also scattered differently at skin inhomogeneities. This can be used to capture skin characteristics. A plurality of photosensors are preferably provided herefor, which can be arranged for example in a detector line along a solid-state light source row, or in the case of only one solid-state light source, along a spatial extent, for example in a longitudinal direction, of the solid-state light source. With such a detector line, the intensity of the back-scattered radiation can be detected. By comparing the respective intensities captured by the photosensors it is possible to determine skin characteristics also in terms of their position and shape, since for example the back-scattered intensity changes if additional scatter media, such as skin inhomogeneities, wounds or the like, are present on the skin surface. Moreover, the photosensors can here be arranged in a plurality of rows, for example in two detector lines, wherein the photosensors of a respective line may be sensitive for different wavelength ranges. This can be done for example with typical detectors having different upstream filters. Owing to these different sensitivity ranges, it is possible to capture skin inhomogeneities and skin characteristics, e.g. also changes in color of a skin area, with significantly higher accuracy by comparing the intensities captured by the respective photosensors, for example by evaluating intensity ratios of the respective captured intensities. Alternatively or additionally to photosensors, the sensor unit can also have other optical capturing devices, although photosensors have the effect that they are particularly cost-effective. It is thus possible with the above-described embodiments of the sensor unit to achieve a particularly simple, cost-effective and yet very precise capturing of skin characteristics.

Various embodiments furthermore relate to a method for operating an irradiation unit for providing radiation pulses for irradiating a surface, wherein the irradiation unit has a light source unit, which is configured to provide the radiation pulses with a specifiable pulse duration, with a specifiable radiant power and at a specifiable temporal pulse spacing, wherein the light source unit is furthermore configured to illuminate a region having a predetermined size at a predetermined distance from the light source unit in a main emission direction of the light source unit. Moreover, the light source unit has at least one solid-state light source, e.g. at least one LED, and the irradiation unit additionally has a control device which drives the at least one solid-state light source. In the case of a plurality of the at least one solid-state light source, the solid-state light sources can be driven individually or in groups by the control device.

The features, feature combinations and effects thereof which are described for the irradiation unit according to various embodiments and the implementations thereof apply equally to the method. Moreover, the objective features mentioned in connection with the irradiation unit and the embodiments thereof make possible the development of the method by way of further method steps.

FIG. 2 shows a schematic of an irradiation unit 20 for providing radiation pulses for irradiating a surface 21 according to an embodiment. The irradiation unit 20 has a light source unit 22 having a plurality of LEDs L₁ to L_{N} arranged in one line. The surface 21 and the irradiation unit 20 are here illustrated with a plan view of the surface 21, that is to say according to the z-direction of the illustrated coordinate system. 21a designates an already treated area of the surface 21, and 21b designates an area of the surface 21 that is yet to be treated. For the treatment, e.g. for hair removal, the irradiation unit is placed on the skin surface such that the light exit opening of the light source unit 22 faces the skin surface. The light source unit 22 can be in direct contact with the skin surface, or spacers may be provided on the irradiation unit 20 which keep the light source unit at a distance from the skin surface, for example at a distance of 0.5 mm to 10 mm. The irradiation unit 20 in this example for illuminating the surface 21 is furthermore moved in the x-direction, while the LEDs L₁ to L_{N} are arranged along the line Z1 in the y-direction. The LEDs L₁ to L_{N} can also be equipped with in each case one primary optics (not illustrated), or a plurality of LEDs can use a common primary optics (not illustrated). When using primary optics, greater distances may be used, for example ranging from 3 mm to 40 mm.

The irradiation unit 20 furthermore has a control device (not illustrated) for driving the LEDs L₁ to L_{N}. By using LEDs, numerous effects can be achieved. In various embodiments, the spectral and temporal emission characteristic of the light source unit 22 can be adapted better to the respective application, such as in the present case hair removal. This furthermore may permit continuous and monitored energy dose introduction into the medium, in the present case the epidermis. However, other uses for the irradiation unit 20 are also conceivable, such as the treatment of vascular lesions and pigment disorders. In addition, the use of LEDs L₁ to L_{N} which emit in the narrow band avoids the necessity of optical filters, which significantly increases the energy efficiency of the irradiation unit 20. In addition, by individually driving the LEDs L₁ to L_{N}, spatial variation of the energy dose is made possible.

The control device is configured to drive the LEDs L₁ to L_{N} in a manner such that they provide light pulses with specifiable radiant power and/or the pulse current height, pulse duration and with specifiable pulse spacing. These control parameters are here set in dependence on the movement speed relative to the surface 21. To this end, the irradiation unit 20 includes a sensor unit 24, which in turn can have a plurality of detectors 24a. In this example, in each case one detector 24a is assigned to a respective LED L₁ to L_{N}. It is possible using the detectors 24a to determine or measure the current displacement speed in the x-direction vₓ=dx/dt, averaged over the movement speed of all LEDs or separately for each individual LED L₁ to L_{N}. This individual speed of a respective LED L₁ to L_{N} is designated v_{x,n} in FIG. 2. The determination of the individual speeds v_{x,n} is here provided, because if the displacement of the treatment head of the irradiation unit 20 takes place along a curved line, this leads to different speeds v_{x,l} and v_{x,r} at the left-hand and right-hand ends of the LED line Z1. This can be taken into consideration in a corresponding driving of the respective LEDs, with result that even in this case uniform energy introduction into the surface 21 is possible. In place of direct ascertainment of the individual speeds v_{x,n} for a respective LED L₁ to L_{N}, it is possible for example to determine only the speed v_{x,l} and v_{x,r} at the respective ends of the LED line Z1, e.g. using mechanical or optical sensors, and the displacement speed v_{x,n} of the individual LEDs L₁ to L_{N} can be interpolated therefrom.

In order to ensure that the same areal energy density is introduced into the area to be treated, the current or the radiant power or the pulse duration for each LED L₁ to L_{N} individually is adapted in a suitable manner in dependence on the respective individual speed v_{x,n}. The same is also true for example if the light source unit 22 has only one solid-state light source. Here, only a single detector 24a may be provided for example, which ascertains the displacement speed v_{x,n} of the solid-state light source.

Provision may furthermore be made for the irradiation unit 20 to inform the user optically or acoustically, for example at the treatment head, about a displacement speed being within the permissible range. If the displacement speed exceeds a first limit value, e.g. a warning is generated. If the displacement speed exceeds, for example, a second limit value which is greater than the first one, an error message is generated and the irradiation unit 20 switches off.

Apart from speed-dependent control of the control parameters, various embodiments also make possible diverse additional further adaptation possibilities. For example, the respective LEDs L₁ to L_{N} can also be controlled in dependence on detected skin inhomogeneities, such as for example a melanocytic nevus 21c. Such melanocytic nevi 21c can here likewise be detected by way of the sensor unit 24, for example using optical sensors, by way of which skin color differences can be identified and the corresponding LEDs, in this example the LED₂, can be switched off or the energy introduction thereof into the skin can be reduced by way of the control parameters pulse height, pulse length or pulse spacing, as soon as the detected melanocytic nevus 21c is within the predetermined illuminable region of the irradiation unit 20. This applies e.g. if the light source unit 22 has only one solid-state light source. Provision of a plurality of solid-state light sources, however, has the effect especially in this case that a region-wise driving of the individual solid-state light sources adapted to the captured position and/or shape of the skin characteristic, such as the melanocytic nevus 21c, but also others, such as wounds, scars, hematomas etc., is possible.

For this purpose, for example a plurality of photosensors can be arranged in the form of a detector line for detecting back-scattered radiation, which is captured by the respective photosensor as an intensity, along the LED lines, or in the case of only one solid-state light source, along a longitudinal extent of the solid-state light source. By evaluating the captured intensity ratios, it is possible to draw conclusions regarding skin characteristics, such as skin color, skin color differences, melanocytic nevus, skin diseases, skin injuries etc., e.g. also regarding the position of such skin characteristics. By providing a plurality of detector lines which are sensitive to different wavelength ranges, it is possible to achieve an even higher accuracy in the detection of such skin characteristics.

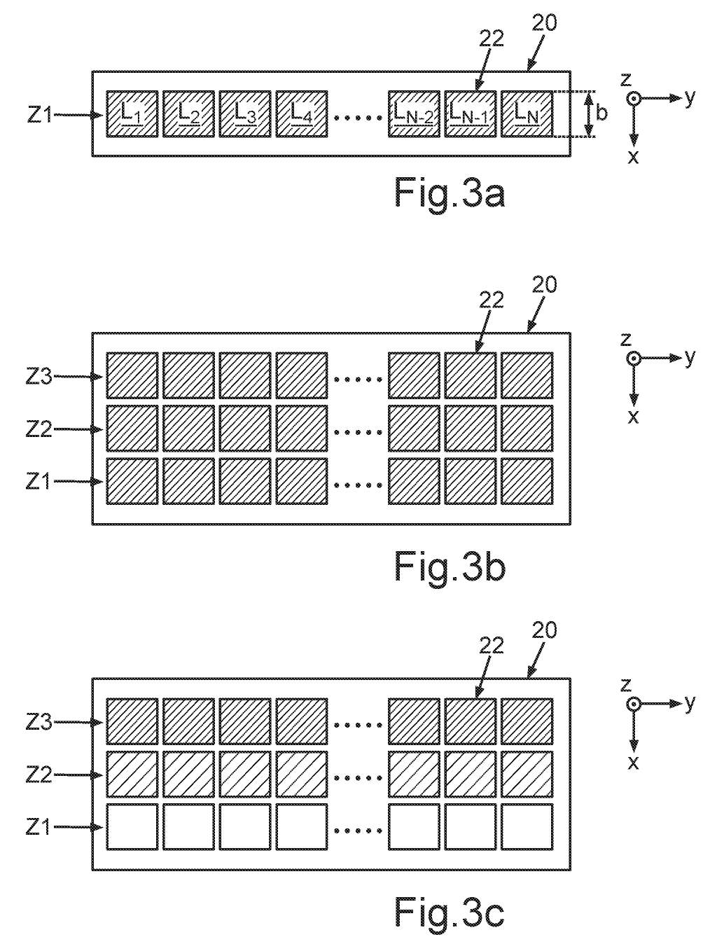

Further embodiments and adaptation possibilities are described in connection with FIG. 3a to FIG. 3c. FIG. 3a here shows a schematic of the irradiation unit 20 having the light source unit 22, which in this example has a plurality of LEDs L₁ to L_{N} arranged in a single line Z1. By way of such a one-dimensional linear LED array, various embodiments can be configured particularly simply and cost-effectively. The chip dimensions of the LEDs L₁ to L_{N} may be approximately 1×1 square millimeters. It is thus possible for example to provide a line, which extends in the y-direction and is approximately 5 cm long, of 50 individual LEDs L₁ to L_{N}. The radiation emitted by the LEDs L₁ to L_{N} can overlap, especially in the case of neighboring LEDs.

However, it is also possible for a plurality of lines to be provided, as illustrated in FIG. 3b. In this example, the irradiation unit 20 has three LED lines Z1, Z2, Z3. Here, each line Z1, Z2, Z3 can also be approximately 5 cm long and be provided with in each case 50 individual LEDs L₁ to L_{N}. Such a two-dimensional LED array arrangement makes possible the shortening of the treatment duration and an increase in the possible displacement speed with the same energy dose.

Additionally, provision may be made for a respective LED line Z1, Z2, Z3 to have LEDs L₁ to L_{N} which differ in terms of their emission wavelength. This is illustrated by way of example in FIG. 3c. Once again, each individual line Z1, Z2, Z3 can have a length of approximately 5 cm and be provided by way of 50 individual LEDs L₁ to L_{N}. The LEDs in the first line Z1 can here have for example an emission wavelength or centroid wavelength of between 800 and 900 nm, the LEDs L₁ to L_{N} of the second line Z2 can have a centroid wavelength of between 950 and 1050 nm, and those of the third line Z3 can have a centroid wavelength of between 1050 and 1150 nm. Typical LEDs here have peak widths at half-height of a few 10 nm, such that even by providing three LED lines having different centroid wavelengths, such as for example in the wavelength ranges stated above, a broad emission spectrum can be provided which thus permits effective hair removal for numerous skin and hair colors. Depending on the hair color or skin color, either only the LEDs of the first line Z1, those of the second line Z2 or those of the third line Z3 may be operated during treatment. It is also possible to operate all lines Z1, Z2, Z3 with different control parameters (pulse height, pulse length, pulse spacing or combinations thereof). The suitable emission wavelength and thus the LED line to be operated may either be selected by the user himself, for example using a corresponding operating element of the irradiation unit 20, or the irradiation unit 20 can independently automatically set the emission wavelength in dependence on the hair and/or skin color detected by the sensor unit 24.

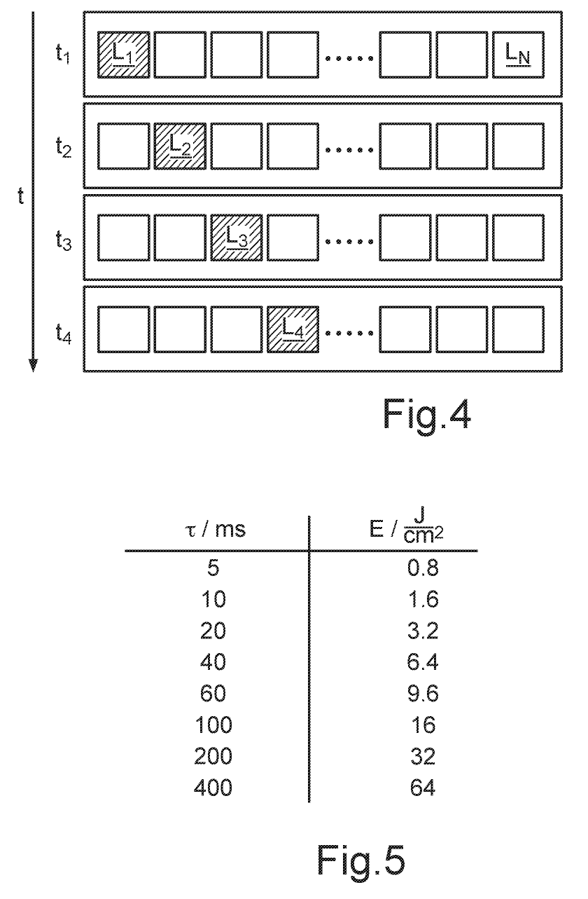

Moreover, provision may be made according to a further embodiment for all LEDs L₁ to L_{N} in a line Z1, Z2, Z3 to be operated at the same time when displacing the treatment head in the x-direction. Since the LEDs L₁ to L_{N} are operated in pulsed fashion, a high pulse power is necessary herefor, however. To lower the necessary pulse power, the LEDs can also be operated in sequence. This is illustrated in FIG. 4, which schematically illustrates the driving of the individual LEDs L₁ to L_{N} of a line in the temporal profile t. In this case, the first LED L₁ emits a light pulse at the time t₁, the second LED L₂ at the time t₂, the third LED L₃ at the time t₃, and the fourth LED L₄ at the time t₄, and so on. If in each case only one LED L₁ to L_{N} is operated per line, the necessary total time for the operation is N×τ, wherein τ is the pulse duration per LED L₁ to L_{N}. In order for the entire area to be treated, it must be ensured that (N×τ)×vₓ<b, wherein b is the width of the LED line, as illustrated in FIG. 3a, in the displacement direction X with displacement speed vₓ.

This sequential driving has the great advantage that the requirements of the maximum necessary pulse power of the power electronics of the irradiation unit 20 can be reduced thereby. If on the other hand a plurality of LEDs L₁ to L_{N} in one line, for example also in groups, are operated at the same time, this has the effect that the displacement speed vₓ can be increased, since in that case (N/Z×τ)×vₓ<b, wherein Z is the number of LEDs which are operated at the same time. If the treatment head consists of a plurality of LED lines, the displacement speed can be increased further and the treatment duration can be correspondingly shortened.

FIG. 5 shows a table for illustrating the connection between pulse duration and areal energy density according to an embodiment. The stated values here relate by way of example to the high-power LED SFH 4715AS. The chip of this LED can provide approximately 1.6 W of average power at a wavelength of 850 nm at real operating temperatures of 50 to 60° C. at a heat sink. This corresponds to a power density of 1.6 W per square millimeter, or 160 W per square centimeter, wherein the LED chip likewise has dimensions of 1×1 square millimeters. In order to achieve a preferred areal energy density of 1 to 10 J per square centimeter, pulse durations of approximately 6 to 60 ms are necessary for this LED. The table in FIG. 5 gives different values for the pulse duration T and the corresponding energy densities E in joules per square centimeter, which are obtained at an average power of 1.6 W. Energy densities of less than 10 J per square centimeter are typically suitable for domestic use, while energy densities of greater than 10 J per square centimeter are generally ascribed to the professional field.

Overall, an irradiation unit having an IPL light source based on LED technology is thus provided, e.g. for hair removal, which offers the possibility of spectral and temporal change in the emission characteristics, and significantly better adaptation to the respective application. It is possible to achieve adaptation of the emission characteristics to the skin type without the necessity of using filters; owing to the spatial variation possibility of the areal energy density, the introduction of a dose that is too high in the region of local variations of pigmentation can be effectively avoided; significantly faster treatment can be provided, since it can be carried out continuously; the efficiency of the irradiation unit can be increased significantly because the entire emission spectrum can be used; and no lamp replacement is necessary since LEDs have a significantly higher lifetime than gas-discharge lamps.

In addition, the irradiation unit is suitable not only for epilation, but for example also for therapeutic treatments of the skin, for example in the case of acne vulgaris, port-wine stains, psoriasis, cellulite, skin discoloration and varicose veins.

### LIST OF REFERENCE SIGNS

10 area
10a segments
20 irradiation unit
21 surface
21a treated area of the surface 21
21b area of the surface 21 yet to be treated
21c melanocytic nevus
22 light source unit
24 sensor unit
24a detectors
b width of the LED line
E energy density
L₁ to L_{N} LEDs
τ pulse duration
t temporal profile
t₁, t₂, t₃ time
v_{x,n} individual speeds
vₓ displacement speed
v_{x,l}, v_{x,r} speeds at the end of the LED line
Z1, Z2, Z3 LED lines

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

What is claimed is:

1. An irradiation unit for providing radiation pulses for irradiating a skin surface, the irradiation unit comprising:
a light source unit configured to provide the radiation pulses with a specifiable pulse duration, with a specifiable pulse height and with a specifiable temporal pulse spacing;
wherein the light source unit is furthermore configured to illuminate a region of the skin surface of a predetermined size at a predetermined distance from the light source unit in a main radiation direction of the light source unit;
wherein the light source unit comprises at least one solid-state light source; and
wherein the irradiation unit further comprises a sensor unit and a control device for driving the at least one solid-state light source.

2. The irradiation unit of claim 1,
configured for providing epilation.

3. The irradiation unit of claim 1,
wherein the at least one solid-state light source represents an LED.

4. The irradiation unit of claim 3,
wherein the light source unit has a plurality of the at least one solid-state light source;
wherein the plurality of solid-state light sources is arranged at least one of along at least one row or along at least one column.

5. The irradiation unit of claim 1,
wherein the light source unit has a plurality of the at least one solid-state light source; and
wherein the control device is configured to drive the solid-state light sources in a specifiable sequence such that the solid-state light sources emit light pulses which are temporally offset from one another.

6. The irradiation unit of claim 1,
wherein the control device is configured to control at least one of the specifiable pulse duration or the specifiable pulse height or the specifiable pulse spacing as at least one control parameter of the at least one solid-state light source.

7. The irradiation unit of claim 6,
wherein the light source unit has a plurality of the at least one solid-state light source and a plurality of light source groups;
wherein each of the light source groups comprises at least one of the plurality of solid-state light sources;
wherein the control device is configured to control the control parameter for a respective light source group separately and independently from one another.

8. The irradiation unit of claim 7,
wherein the control device is configured to control the control parameter for a respective solid-state light source of the plurality of solid-state light sources separately and independently from one another.

9. The irradiation unit of claim 7,
wherein the sensor unit is configured to capture a sensor variable which relates to a movement of the irradiation unit relative to the skin surface; and
wherein the control unit is configured for controlling the control parameter in dependence on the captured sensor variable.

10. The irradiation unit of claim 9,
wherein the captured sensor variable at least one of represents a movement speed with which the irradiation unit is moved relative to the surface or represents a path distance over which the irradiation unit is moved relative to the surface.

11. The irradiation unit of claim 1,
wherein the light source unit has a plurality of the at least one solid-state light source;
wherein the sensor unit is configured to capture the sensor variable with respect to two different locations of the light source unit as a first sensor variable and a second sensor variable; and
wherein the sensor unit is further configured to ascertain from the first and second sensor variables an associated individual sensor variable for a respective light source group or for a respective solid-state light source;
wherein the control device is configured to control the control parameter of the respective light source group or the respective solid-state light source in dependence on the assigned individual sensor variable.

12. The irradiation unit of claim 10,
wherein at least one specifiable limit value is specified for the movement speed; and
at least one of wherein the irradiation unit is configured to output a warning signal to a user if the limit value is exceeded, or wherein the control device is configured to switch off the irradiation unit if the limit value is exceeded.

13. The irradiation unit of claim 1,
wherein the light source unit has a plurality of the at least one solid-state light source and at least one first solid-state light source of the solid-state light sources has a first emission spectrum, and at least one second solid-state light source of the solid-state light sources has a second emission spectrum, which differs from the first one.

14. The irradiation unit of claim 13,
wherein the first centroid wavelength has a first centroid wavelength, and
wherein the second emission spectrum, which differs from the first one, has a second centroid wavelength, which differs from the first one.

15. The irradiation unit of claim 1,
wherein the control device is configured to set an emission wavelength of the light source unit in dependence on a user input captured via an operating element of the irradiation unit.

16. The irradiation unit of claim 1,
wherein the sensor unit is configured to capture a skin characteristic of the skin surface; and
wherein the control device is configured to control at least one solid-state light source in dependence on the captured skin characteristics for irradiating the region.

17. The irradiation unit of claim 16,
wherein the skin characteristic represents a skin characteristic which is located at least partially within the region of the skin surface;
wherein the sensor unit is configured to capture the skin characteristic before the irradiation of the region, in terms of time.

18. The irradiation unit of claim 16,
wherein the sensor unit is configured at least one of to capture a position of the skin characteristic within the region or to capture a shape of the skin characteristic.

19. The irradiation unit of claim 16,
wherein the light source unit has a plurality of the at least one solid-state light source and the control device is configured to drive the solid-state light sources such that the region is irradiated with an irradiation characteristic, which varies spatially over the region in dependence on the captured skin characteristic.

20. A method for operating an irradiation unit for providing radiation pulses for irradiating a skin surface, the method comprising:
a light source unit providing the radiation pulses with a specifiable pulse duration, with a specifiable pulse height and with a specifiable temporal pulse spacing;
the light source unit furthermore illuminating a region of the skin surface of a predetermined size at a predetermined distance from the light source unit in a main radiation direction of the light source unit;
wherein the light source unit has at least one solid-state light source and the irradiation unit has a sensor unit and a control device;
the control device driving the at least one solid-state light source.
